(19) United States
(12) Patent Application Publication
(10) Pub. No.: US 2026/0083907 A1
(43) Pub. Date: Mar. 26, 2026

(54) INTRAVENOUS NEEDLE INSERTION DEVICE

(71) Applicant: Temple University - Of The Commonwealth System of Higher Education, Philadelphia, PA (US)

(72) Inventors: Eric Gokcen, Langhorne, PA (US); Cem Gokcen, Greenville, OH (US)

(21) Appl. No.: 19/112,636

(22) PCT Filed: Sep. 19, 2023
(86) PCT No.: PCT/US2023/074517

§ 371 Date: Mar. 18, 2025

Related U.S. Application Data
(60) Provisional application No. 63/408,806, filed on Sep. 21, 2022.
(60) Provisional application No. 63/499,290, filed on May 1, 2023.

(51) Int. Cl. A61M 5/158

(52) U.S. Cl. CPC A61M 5/158; A61M 2005/1585; A61M 2205/58; A61M 2209/088; A61M 2210/12

(57) ABSTRACT

An intravenous needle insertion device is described. The device includes a body having a front end, a back end, a bottom surface and a top surface sloping downward toward the front end. The device further includes a vein trough recessed within the bottom surface of the body, a needle trough recessed within the top surface of the body, a puncture window at the front end of the body, where the vein trough and the needle trough both lead into the puncture window.

### CROSS-REFERENCE TO RELATED APPLICATIONS

This application claims the benefit of priority to U.S. Patent Application No. 63/499,290, filed on May 1, 2023, and U.S. Provisional Application No. 63/408,806, filed on Sep. 21, 2022, the contents of which are each incorporated by reference herein as if set forth in their entirety.

### BACKGROUND OF THE INVENTION

Intravenous (IV) catheters and needles are used for numerous medical procedures to access patient veins. However, the insertion of the catheters and needles into patient veins can be difficult, painful, and possibly anxiety provoking to both the healthcare provider and the patient. For example, unstable or rolling veins may cause the vein to move away from the needle right at the time of attempted puncture which could result in a miss or a partial insertion, thus further resulting in a hematoma. Also, an additional puncture attempt may be necessary and may even require several attempts before being successful, causing even more pain and damage to the patient and their veins. Furthermore, the healthcare provider may not even be able to successfully insert the IV catheter at all, requiring more invasive procedures to obtain the necessary IV catheter or needle access. In addition, safety is always a concern when using sharp medical instruments. When attempting to insert an IV catheter or needle, the healthcare provider often needs to stabilize the vein with their non-dominant hand, close to the insertion site; this may increase the risk of an accidental needle stick into the hand.

Thus, there is a need in the art for a device that can stabilize the vein and guide the IV needle directly into the patient's vein to minimize the anxiety and maximize the safety of the patient and healthcare provider. The present invention satisfies that need.

### SUMMARY OF THE INVENTION

An intravenous needle insertion device is described. The device includes a body having a front end, a back end, a bottom surface and a top surface sloping downward toward the front end. The device further includes a first trough recessed within the bottom surface of the body, a second trough recessed within the top surface of the body, a puncture window at the front end of the body, where the first trough and the second trough both lead into the puncture window. In some embodiments, the body is wedge-shaped. In some embodiments, the slope of the top surface relative to the bottom surface is at an angle of between 10 and 45 degrees. In some embodiments, the puncture window is flanked by two tines defining the puncture window by their opposing inside walls. In some embodiments, the first trough is configured to stabilize a subject's vein when the subject's vein is positioned within the first trough. In some embodiments, the width of the puncture window is equal to or greater than the width of the first trough. In some embodiments, the width of the first trough is between 2 and 7 millimeters. In some embodiments, the width of the second trough is between 0.5 and 5 millimeters. In some embodiments, the width of the puncture window is between 1 and 7 millimeters. In some embodiments, the second trough is aligned with the center of the puncture window. In some embodiments, the device is composed of a rigid material. In some embodiments, the device further includes at least two pressure points.

Also described is a needle insertion device including the features of a wedge-shaped body having a bottom surface and a top surface that slopes downward toward a front end, a trough recessed within the bottom surface of the body, a needle guide on or in the top surface of the body, and a puncture window at the front end of the body where the trough leads into the puncture window. In some embodiments, the slope of the top surface relative to the bottom surface is at an angle of between 10 and 45 degrees. In some embodiments, the puncture window is flanked by two tines defining the puncture window by their opposing inside walls. In some embodiments, the trough is configured to stabilize a subject's vein when the subject's vein is positioned within the trough. In some embodiments, the width of the puncture window is equal to or greater than the width of the trough. In some embodiments, the needle guide is a groove recessed within the top surface of the body. In some embodiments, the needle guide is aligned with the center of the puncture window.

Also described is an intravenous needle insertion device having a wedge-shaped body with a front end, a back end, a bottom surface and a top surface sloping downward toward the front end at an angle between 10 and 45 degrees, a puncture window at the front end of the body, a vein trough recessed within the bottom surface of the body and leading into the puncture window, and a needle trough recessed within the top surface of the body and leading into the puncture window where the needle trough and the vein trough are in line with each other.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1A is a top view of an exemplary intravenous needle insertion device.

FIG. 1B is a bottom view of the exemplary intravenous needle insertion device from FIG. 1A.

FIG. 1C is a side view of the exemplary intravenous needle insertion device from FIGS. 1A and 1B.

FIG. 2A is a top perspective view of an exemplary 3-D printed intravenous needle insertion device.

FIG. 2B is a bottom perspective view of the exemplary 3-D printed intravenous needle insertion device of FIG. 2A.

FIG. 3 is a schematic of a top view of another exemplary intravenous needle insertion device.

FIGS. 4A-4C are a schematic of another exemplary intravenous needle insertion device that utilizes an exemplary strap. FIG. 4A shows a top view of the device without a strap. FIG. 4B shows a top view of the device with an exemplary strap. FIG. 4C shows a side view of the device with the exemplary strap positioned for adjustable securement around a subject's wrist.

FIG. 5 is an image of the exemplary intravenous needle insertion device from FIGS. 2A and 2B in use.

### DETAILED DESCRIPTION OF THE INVENTION

It is to be understood that the figures and descriptions of the present invention have been simplified to illustrate elements that are relevant for a clearer comprehension of the present invention, while eliminating, for the purpose of clarity, many other elements found in systems and methods of intravenous needle insertion device. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Ranges: throughout this disclosure, various aspects of the invention can be presented in a range format. It should be understood that the description in range format is merely for convenience and brevity and should not be construed as an inflexible limitation on the scope of the invention. Where appropriate, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Referring now in detail to the drawings, in which like reference numerals indicate like parts or elements throughout the several views, in various embodiments, presented herein is an intravenous needle insertion device.

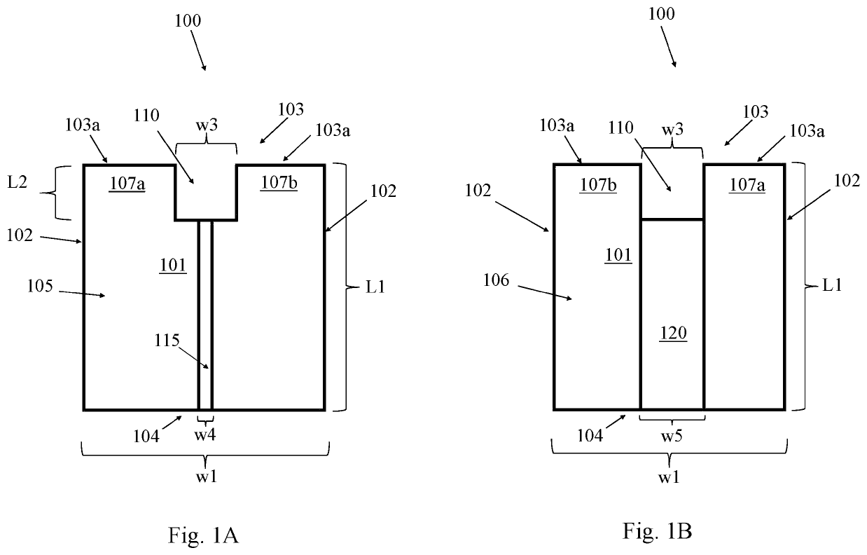

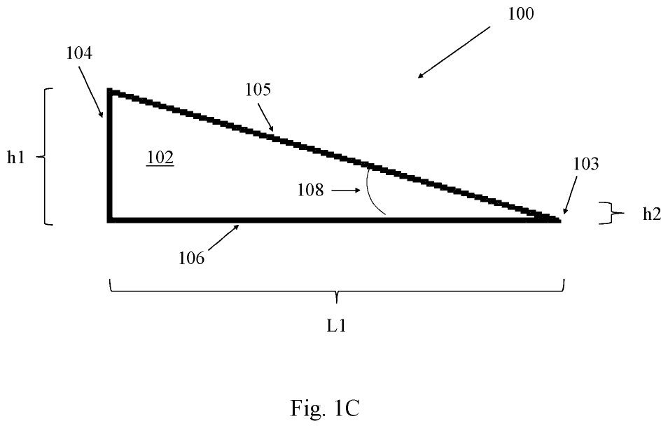

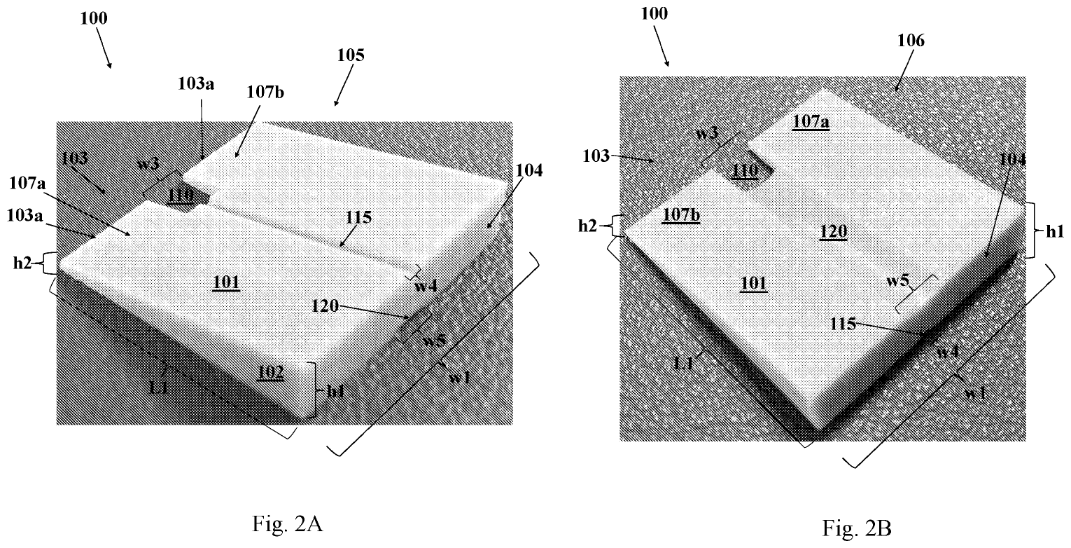

Referring now to FIGS. 1A-1C and 2A-2B, an exemplary intravenous needle insertion device 100 is presented. As contemplated herein, an intravenous needle insertion device may be used to hold, trap or otherwise stabilize a vein of a patient in order for a medical professional to easily access the vein with a needle, catheter, or other device used to puncture veins at a desired angle. Device 100 includes a body 101 which is generally wedge-shaped with a top surface 105 that slopes downwards to a leading edge 103a at a front end 103 of body 101. Body 101 further includes a flat or substantially flat bottom surface 106, with top surface 105 sloping at an angle 108 relative to bottom surface 106. Body 101 further includes a back end 104 and side walls 102. Body 101 may be any shape about its perimeter (i.e. top down view), such as, without limitation, square, rectangular, trapezoid, curved, oval or the like.

As shown in FIGS. 1A-1B and 2A-2B, body 101 of device 100 has a width w1, which represents the distance between opposing side walls 102. Device width w1 may be between 10 mm and 40 mm. Device 100 further includes a length L1 representing the distance between the front end 103 and back end 104. Device length L1 may be between 10 mm and 40 mm.

As shown in FIG. 1C and mentioned previously, body 101 of device 100 is wedge-shaped with its top surface 105 sloping downward toward leading edge 103a. Accordingly, device 100 has a back-end height h1 representing the distance between bottom surface 106 and top surface 105 at back end 104. Back-end height h1 may be between 2 mm and 10 mm. Similarly, device 100 has a front-end height h2 representing the distance between bottom surface 106 and top surface 105 at front end 103, terminating at leading edge 103a. Front-end height h2 may be between 0 mm and 3 mm. In some embodiments, leading edge 103a may terminate at a point and does not have an appreciable height h2. In other embodiments, leading edge 103a may have a front vertical face of height h2.

As shown in FIGS. 1B and 2B, bottom surface 106 is generally flat or planar. In some embodiments, bottom surface 106 may be curved or contoured, provided that bottom surface 106 is suitable for placement against a subject's skin. In some embodiments, bottom surface 106 may include an adhesive to hold or assist in holding device 100 in place on the subject's skin. Bottom surface 106 includes one or more grooves or troughs 120 running along all or a portion of the length of body 101. Trough 120 is recessed within bottom surface 106 and generally runs in a straight line along the body length. In some embodiments, trough 120 may include one or more turns along its length. The recess formed by trough 120 in bottom surface 106 may be any depth and may further be rectangular, V-shaped, curved or semi-circular, or any other cross-sectional shape desired. In some embodiments, trough 120 has an average depth of 1 to 5 mm. Trough 120 has a width w5 of between 2 and 7 mm. As contemplated herein, trough 120 is sized and shaped to permit a subject's vein to fit within trough 120 when bottom surface 106 is pressed overtop a region of the subject's skin containing the vein. By doing so, the subject's vein becomes stabilized within trough 120 and consequently further stabilized on either side of the front and back ends 103, 104 of body 101. As such, the subject's vein is not capable of sliding or rolling underneath the patient's skin when positioned within trough 120. In some embodiments, the width w5 of trough 120 may be adjustable to adequately stabilize different sizes of veins.

As shown in FIGS. 1A-1B and 2A-2B, body 101 may further include tines 107a and 107b flanking a puncture window 110. Tines 107a and 107b form leading edge 103a at their front ends, and further form puncture window 110 between their inside walls. Puncture window 110 is generally in-line with trough 120, such that trough 120 leads into puncture window 110 at front end 103 of body 101. In this manner, when a subject's vein is stably positioned within trough 120, the vein likewise runs through puncture window 110 and remains stabilized between the inside walls of tines 107a and 107b. In some embodiments, tines 107a and 107b have opposing inside walls that are parallel to each other. In some embodiments, tines 107a and 107b may have a gap between them that is V-shaped to accommodate smaller veins.

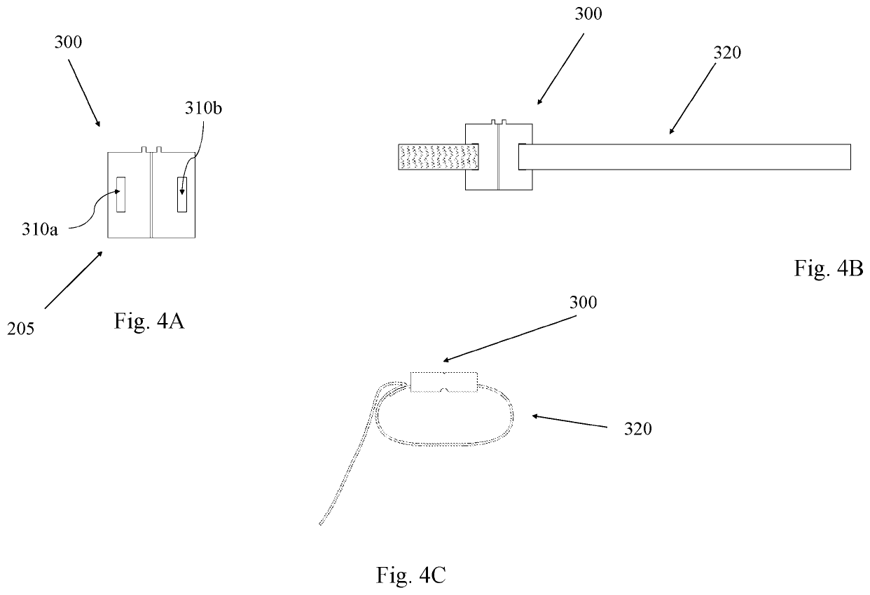

Puncture window 110 has a width w3 that may be equal to or greater than width 5 of trough 120. In some embodiments, width w3 may be between 1 mm and 7 mm. Puncture window 110 further has a length L2 formed by the inside walls of tines 107a and 107b. In some embodiments, length L2 may be between 2 mm and 10 mm. As contemplated herein, the length L2 of puncture window 110 should permit for the puncturing of the subject's vein within the window when a needle is positioned into device 100, as described below and as shown in FIG. 4.

As shown in FIGS. 1A and 2A, top surface 105 is generally flat or planar. Top surface 105 includes one or more grooves or troughs 115 running along all or a portion of the length of body 101 and leads into puncture window 110. As such, trough 115 functions as a needle guide. Trough 115 is recessed within top surface 105 and generally runs in a straight line along the body length. The recess formed by trough 115 in top surface 105 may be any depth and may further be rectangular, V-shaped, curved or semi-circular, or any other cross-sectional shape desired. In some embodiments, trough 115 has an average depth of 0.5 to 5 mm. Trough 115 has a width w4 of between 0.5 and 5 mm. As contemplated herein, trough 115 is sized and shaped to permit a needle shaft to fit within trough 115 and guide the needle securely toward puncture window 110. In some embodiments, trough 115 is centrally aligned with trough 120 and puncture window 110, such that when a needle is positioned in trough 115, the needle tip is positioned in the central or mid-line of puncture window 110. In some embodiments, top surface 105 may include one or more securement elements configured to further secure a needle positioned within trough 115.

Referring again to FIG. 1C, top surface 105 is sloped downward toward leading edge 103a at an angle 108 relative to bottom surface 106. Angle 108 may be between 10° and 45°. In some embodiments, angle 108 is equal to or greater than 10°. In some embodiments, angle 108 is equal to or less than 45°. In some embodiments, angle 108 is 12°. In some embodiments, angle 108 is 17°. In some embodiments, angle 108 is 27°. It should be appreciated that angle 108 determines the trajectory of the needle when positioned in trough 115 and guided towards a vein stabilized within puncture window 110.

Device 100 may be composed of any sturdy or rigid material suitable for medical equipment, such as, without limitation, plastic, metal, carbon fiber, wood or any similar material. Device 100 may be constructed via any traditional manufacturing technique, including 3-D printing, molding, multi-part assembly and the like. The composition of device 100 may be selected for single use applications, or it may be selected for ease of washing and/or sterilization for repeated use.

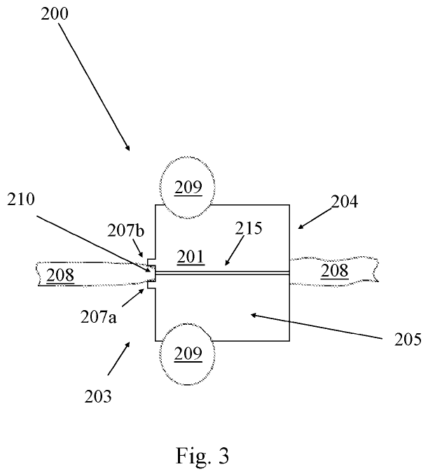

FIG. 3 illustrates another exemplary embodiment of an intravenous needle insertion device 200 engaging with a subject's vein 208. Device 200 comprises body 201, side walls 202, front end 203, back end 204, top surface 205, a bottom surface with a recessed vein trough, first tine 207a, second tine 207b, puncture window 210, and needle trough 215. First tine 207a and second tine 207b may be any shape or size as long as the inside walls stabilize the subject's vein. Also shown in FIG. 3 are pressure points 209. As contemplated herein, a user may apply downward pressure with their fingers to both hold device 200 against the subject's skin and also to help present the stabilized vein positioned within the vein trough in the bottom surface and within the puncture window 210. Pressure points 209 may be located anywhere on top surface 205 and/or on body 201.

FIGS. 4A-C illustrate another exemplary embodiment of an intravenous needle insertion device 300 with a strap. In a nonlimiting manner, device 300 may include any device features as described herein for other embodiments, and likewise, all other embodiments may similarly include a strap. Exemplary device 300 may include open slots 310a and 310b which may be holes, apertures, slits, or the like to allow a strap 320 to be fitted through. In various embodiments, strap 320 may be a thick or thin material that is flexible and/or elastic. Strap 320 may be releasably securable around at least a portion of a subject's body, such as around the hand, wrist, forearm elbow or upper arm. Strap 320 may be adjustable in size to fit variable sized circumference, and at relative amounts of force or tightness. The releasable securement mechanism of device 300 may include any standard components such as a hook-and-loop or hook-and-pile fastener on strap 320, adhesive surfaces on strap 320, buckle mechanisms, snaps, and the like.

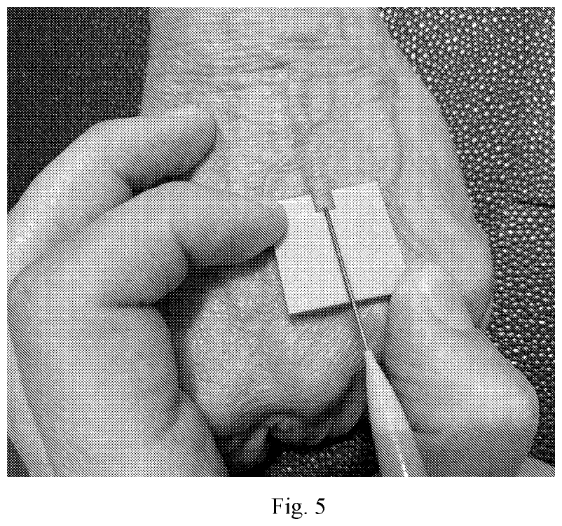

FIG. 5 shows exemplary device 100 in use. The vein to be accessed is identified, and the bottom surface of device 100 is placed overtop the vein, such that the vein is positioned within the vein trough 120 and puncture window 110. With the subject's vein in place, the user applies a slight downward pressure on device 100 to confirm the vein is stabilized and presented within the puncture window. A needle is then positioned in the needle trough 115 and slide forward until the needle tip punctures the vein at the desired depth in puncture window 110. The needle can then be subsequently retracted when access to the vein is no longer needed.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention.

What is claimed is:

1. An intravenous needle insertion device, comprising:
a body having a front end, a back end, a bottom surface and a top surface sloping downward toward the front end;
a first trough recessed within the bottom surface of the body;
a second trough recessed within the top surface of the body; and
a puncture window at the front end of the body;
wherein the first trough and the second trough lead into the puncture window.

2. The device of claim 1, wherein the body is wedge-shaped.

3. The device of claim 1, wherein the slope of the top surface relative to the bottom surface is at an angle of between 10 and 45 degrees.

4. The device of claim 1, wherein the puncture window is flanked by two tines defining the puncture window by their opposing inside walls.

5. The device of claim 1, wherein the first trough is configured to stabilize a subject's vein when the subject's vein is positioned within the first trough.

6. The device of claim 1, wherein the width of the puncture window is equal to or greater than the width of the first trough.

7. The device of claim 1, wherein the width of the first trough is between 2 mm and 7 mm.

8. The device of claim 1, wherein the width of the second trough is between 0.5 mm and 5 mm.

9. The device of claim 1, wherein the width of the puncture window is between 1 mm and 7 mm.

10. The device of claim 1, wherein the second trough is aligned with the center of the puncture window.

11. The device of claim 1, wherein the device is composed of a rigid material.

12. The device of claim 1, further comprising at least two pressure points.

13. An intravenous needle insertion device, comprising:
a wedge shaped body having a bottom surface and a top surface that slopes downward toward a front end;
a trough recessed within the bottom surface of the body;
a needle guide on or in the top surface of the body; and
a puncture window at the front end of the body;
wherein the trough leads into the puncture window.

14. The device of claim 13, wherein the slope of the top surface relative to the bottom surface is at an angle of between 10 and 45 degrees.

15. The device of claim 13, wherein the puncture window is flanked by two tines defining the puncture window by their opposing inside walls.

16. The device of claim 13, wherein the trough is configured to stabilize a subject's vein when the subject's vein is positioned within the trough.

17. The device of claim 13, wherein the width of the puncture window is equal to or greater than the width of the trough.

18. The device of claim 13, wherein the needle guide is a groove recessed within the top surface of the body.

19. The device of claim 13, wherein the needle guide is aligned with the center of the puncture window.

20. An intravenous needle insertion device, comprising:
a wedge-shaped body having a front end, a back end, a bottom surface and a top surface sloping downward toward the front end at an angle between 10 and 45 degrees;
a puncture window at the front end of the body;
a vein trough recessed within the bottom surface of the body and leading into the puncture window; and
a needle trough recessed within the top surface of the body and leading into the puncture window;
wherein the needle trough and the vein trough are in line with each other.

21. The device of claims 1, 13 or 20, further comprising a strap configured to releasably secure the device to a surface of a subject's body.
